(19) United States
(12) Patent Application Publication
(10) Pub. No.: US 2006/0018838 A1
(43) Pub. Date: Jan. 26, 2006

(54) Vacsularized tissue for transplantation

(75) Inventors: Steven C George, Irvine, CA (US); Noo Li Jeon, Irvine, CA (US); Chris C.W. Hughes, Irvine, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: THE REGENTS OF THE UNIVERSITY OF CALIFORNIA, OAKLAND, CA (US)

(21) Appl. No.: 10/507,652

(22) PCT Filed: Mar. 12, 2003
(86) PCT No.: PCT/US03/07720

§ 371 Date: Sep. 10, 2004

Related U.S. Application Data
(60) Provisional application No. 60/363,665, filed on Mar. 12, 2002.

(51) Int. Cl. A61K 48/00; A61L 9/04

(52) U.S. Cl. 424/44; 424/93.7

(57) ABSTRACT

Tissue engineering holds enormous potential to replace or restore function to a wide range of tissues. However, the most successful applications have been limited to thin avascular tissues in which delivery of essential nutrients occurs primarily by diffusion. Pursuant to the present invention, a prevascularized, thick tissue construct is created having a network of capillaries with lumens capable of nutrient and origin delivery and forming anastamoses to host vasculature. A tissue transplantation strategy is comprised of (1) in vitro vascularization of a tisue construct, (2) transplantation of prevascularized tissue to wound bed of host where vessels of implantable tissue and host rapidly anastomose, and (3) host-directed remodeling and reorganization of the tissue and vascular network.

### FIELD OF THE INVENTION

This application relates to engineered tissue having an engineered vascular network for forming anastamoses to endogenous vasculature after transplantation and methods to produce the tissue in vitro.

### BACKGROUND

Tissue transplantation is critically necessary in many clinical situations, including reconstructive surgery, wound healing, cardiovascular treatment and many others. The first examples of tissue trasplantation were “autologous,” meaning that the tissue was simply removed from a donor site in the patient and then re-inserted at another target site. Although autologous tissue transfers are well known, autologous transfers have certain drawbacks that cannot be overcome. Significantly, autologous transfers compromise the donor site and carry the risk of infection and loss of function. In a surgical setting, a second procedure to remove an autologous tissue graft always carries a finite risk and unavoidably adds to patient discomfort and expense.

Many of the problems inherent in endogenous transplants could be overcome by transplanting exogenous tissue or by a useable synthetic tissue replacement. Beginning with the first examples of surgical tissue transplants, physicians, researchers and medical scientists of all kinds have been searching for techniques to engineer tissue to permit successful transplantation to a target site within a patient. Several approaches to synthetic tissues have been pursued, including polymers and mixtures of natural tissue and synthetic substrates, but with limited success. In vitro engineering of human tissue has also been performed; however, the size of the transplant, in both surface area and volume, is limited by the ability of tissue grown in an in vitro culture system to sustain a vascular network. A vascular network is necessary in any tissue segment above a certain size to maintain the flow of oxygen and to deliver nutrients. Fabricating a tissue construct in vitro with sufficient vascularization is particularly difficult because such a construct has exacting requirements based on each of vascular biology, material science, microfabrication, mass transfer, and a clinical perspective on the implantable tissue.

To date, success in tissue engineering has been in avascular tissues, that are relatively thin (thickness <2 mm) in which the supply of nutrients and oxygen is primarily by a diffusion mechanism across all membranes. Avascular examples include the epidermis of skin which has received FDA approval, and cartilage such as the nasal septae. More complex tissues such as cardiac muscle and liver have been attempted but have been limited to thin (<70 microns) sections. More homogeneous tissues such as adipose tissue and smooth muscle have been met with some success but have also been limited to dimensions <2.5 mm. Bulkier soft tissues for reconstructive surgery have proved more difficult due to the need for an immediate vascular supply to maintain the tissue after the transplant is performed.

New blood vessel growth, neovascularization, can be categorized as the growth of new vessels from existing vessels (angiogenesis), or the development of new vessels from progenitor cells (vasculogenesis). Angiogenesis is most commonly observed in tumor growth, wound healing, and the female reproductive cycle, whereas vasculogenesis is observed in embryogenesis, Breier G., “Angiogenesis in embryonic development—a review,” Placenta 21 Suppl A: S11-15, 2000 and Breier G, Albrecht U, Sterrer S and Risau W., “Expression of vascular endothelial growth factor during embryonic angiogenesis and endothelial cell differentiation,” Development 114: 521-532, 1992. Neovascularization is stimulated by a series of soluble proteins commonly referred to as angiogenic factors. Vascular endothelial growth factor (VEGF) and basic fibroblast growth factor (bFGF) are direct angiogenic factors as they directly stimulate the change in the endothelial cells from a quiescent to a proliferative and migratory phenotype. Other important indirect angiogenic growth factors include platelet-derived growth factor (PDGF) Soker S, Machado M and Atala A., “Systems for therapeutic angiogenesis in tissue engineering,” World J Urol 18: 10-18, 2000, transforming growth factor-B (TGF-B) Pepper M S., “Transforming growth factor-beta: vasculogenesis, angiogenesis, and vessel wall integrity,” Cytokine Growth Factor Rev 8: 21-43, 1997, and the angiopoietins (Ang) Hanahan D., “Signaling vascular morphogenesis and maintenance,” Science 277: 48-50, 1997.

VEGF is a family of five homodimeric proteins (VEGF A, B, C, D, and PDGF) that are potent regulators of neovascularization, Ferrara N and Davis-Smyth T., “The biology of vascular endothelial growth factor,” Endocr Rev 18: 4-25, 1997. They are specific mitogens for endothelial cells, and also stimulate endothelial cells to migrate and form tubes. There are five isoforms of VEGF A, which vary in their pattern of expression and localization, but VEGF₁₂₁ and VEGF₁₆₅ are the most abundant isoforms and the two that are soluble. The remaining isoforms are primarily localized to the cell surface, Soker S, Machado M and Atala A., “Systems for therapeutic angiogenesis in tissue engineering,” World J Urol 18: 10-18, 2000. The expression of VEGF is greatly enhanced in tissues undergoing vascularization in vivo, and is also stimulated by hypoxia, Helmlinger G, Endo M, Ferrara N, Hlatky L and Jain R K., “Formation of endothelial cell networks,” Nature 405: 139-141, 2000 and Shima D T, Deutsch U and D'Amore P A., “Hypoxic induction of vascular endothelial growth factor (VEGF) in human epithelial cells is mediated by increases in mRNA stability,” FEBS Lett 370: 203-208, 1995. bFGF is a potent angiogenic factor, and is a mitogen for both EC as well as fibroblasts, Abraham J A, Mergia A, Whang J L, Tumolo A, Friedman J, Hjerrild K A, Gospodarowicz D and Fiddes J C., “Nucleotide sequence of a bovine clone encoding the angiogenic protein, basic fibroblast growth factor,” Science 233: 545-548, 1986. Its expression is distributed widely in both normal and pathologic tissues, and it also plays a critical role in wound healing by stimulating re-epilthelialization and angiogenesis. Both VEGF and bFGF are routinely used in models of angiogenesis including those related to engineering vascularized tissues.

More recently, it has been shown that an in vitro co-culture of stromal cells and ECs will form a capillary network that can be stable for up to 50 days Frerich B, Lindemann N, Kurtz-Hoffmann J and Oertel K., “In vitro model of a vascular stroma for the engineering of vascularized tissues,” Int J Oral Maxillofac Surg 30: 414-420, 2001. This tissue received nutrients through diffusion and was thus limited to a total volume of <0.5 ml. Schechner J S, Nath A K, Zheng L, Kluger M S, Hughes C C, Sierra-Honigmann M R, Lorber M I, Tellides G, Kashgarian M, Bothwell A L and Pober J S., “In vivo formation of complex microvessels lined by human endothelial cells in an immunodeficient mouse,” Proc Natl Acad Sci USA 97: 9191-9196, 2000, have recently reported conditions in which preformed endothelial tubes in a collagen-fibrin gel became integrated into the host vasculature upon implanation in mice. The tissues were approximately 2 mm thick, and the endothelial tubes were stabilized by overexpressing Bcl-2 in the ECs. Thus, a preformed capillary network of sufficient size and dimension is capable of becoming integrated into the host vasculature upon implantation. Presently, three approaches exist for designing vascularized engineered tissues: 1) implanting avascular tissues with biochemical factors to stimulate angiogenesis, the rapid ingrowth of vessels in vivo; 2) seeding porous implantable biodegradable polymer scaffolds, with or without endothelial cells, to provide bulk and stimulate vessel formation in vivo, and 3) prevascularizing artificial tissue prior to cell seeding or implantation.

The first approach includes degradable microcarriers and cellular transfection. Degradable microcarriers can be used to release angiogenic growth factors such as VEGF or bFGF, Cleland J L, Duenas E T, Park A, Daugherty A, Kahn J, Kowalski J and Cuthbertson A., “Development of poly-(D,L-lactide-coglycolide) microsphere formulations containing recombinant human vascular endothelial growth factor to promote local angiogenesis,” J Control Release 72: 13-24, 2001; Elcin Y M, Dixit V and Gitnick G., “Extensive in vivo angiogenesis following controlled release of human vascular endothelial cell growth factor: implications for tissue engineering and wound healing,” Artif Organs 25: 558-565, 2001; Hopkins S P, Bulgrin J P, Sims R L, Bowman B, Donovan D L and Schmidt S P, “Controlled delivery of vascular endothelial growth factor promotes neovascularization and maintains limb function in a rabbit model of ischemia,” J Vasc Surg 27: 886-894; discussion 895, 1998; King T W and Patrick C W, Jr., “Development and in vitro characterization of vascular endothelial growth factor (VEGF)-loaded poly(DL-lactic-co-glycolic acid)/poly(ethylene glycol) microspheres using a solid encapsulation/single emulsion/solvent extraction technique,” J Biomed Mater Res 51: 383-390, 2000; (Ferrara, N. et al., “The biology of vascular endothelial growth factor, Endocr Rev 18: 4-25, 1997; Freed, L. E., et al., “Biodegradable polymer scaffolds for tissue engineering,” Biotechnology (NY) 12: 689-693, 1994; Li, R. K. et al., “Construction of a bioengineered cardiac graft,” J Thorac Cardiovasc Surg 119: 368-375, 2000; Tabata Y, Miyao M, Ozeki M and Ikada Y., “Controlled release of vascular endothelial growth factor by use of collagen hydrogels,” J Biomater Sci Polym Ed 11: 915-930, 2000.

For example, degradable polymeric microspheres (poly-(D,L-lactide-coglycolide)) release recombinant human VEGF in a controlled fashion and stimulate new vessel growth in a dose dependent fashion, Cleland J L, ET AL., supra. Alternately, a mammalian cell can be transfected with a DNA construct to overexpress an angiogenic cofactor. Ajioka I, Nishio R, Ikekita M, Akaike T, Sasaki M, Enami J and Watanabe Y, “Establishment of heterotropic liver tissue mass with direct link to the host liver following implantation of hepatocytes transfected with vascular endothelial growth factor gene in mice,” Tissue Eng 7: 335-344, 2001. For example, chinese hamster ovary (CHO) cells transfected with VEGF₁₆₅ cDNA, and then encapsulated in Ca-alginate poly-L-lysine microspheres, have been shown to increase vascularization near the implantation site of a cell-seeded matrix in mice, Soker S, Machado M and Atala A., “Systems for therapeutic angiogenesis in tissue engineering,” World J Urol 18: 10-18, 2000. The primary drawback of this approach is that the time needed for ingrowth of new vessels from the host following the transplant may exceed the ability of the tissue to survive without the transport of oxygen and the flow of nutrients.

The second approach involves the use of degradable polymer scaffolds that can provide bulk and porosity for a transplanted tissue construct and can also encourage the ingrowth of vessels in vivo. An early study utilized a degradable PGA scaffold seeded with chrondrocytes and demonstrated tissue differentiation, but was limited to a thickness of 0.35 cm. Freed L E, Vunjak-Novakovic G, Biron R J, Eagles D B, Lesnoy D C, Barlow S K and Langer R, “Biodegradable polymer scaffolds for tissue engineering,” Biotechnology (N Y) 12: 689-693, 1994. More recently, a macroporous hydrogel bead using sodium alginate covalently coupled with an arginine, glycine, and aspartic acid-containing peptide was demonstrated to maintain bulk and induce the ingrowth of vessels six months post-implant in mice. Kaihara S, Borenstein J, Koka R, Lalan S, Ochoa E R, Ravens M, Pien H, Cunningham B and Vacanti J P, “Silicon micromachining to tissue engineer branched vascular channels for liver fabrication,” Tissue Eng 6: 105-117, 2000. The size of these implantable beads ranged from 2.7-3.2 mm in diameter. If the implantable scaffold contains cells of a specific phenotype (i.e., hepatocytes or cardiac myocytes), the primary disadvantage, as with the first approach, is the reliance on diffusion to deliver nutrients and oxygen while waiting for the ingrowth of new vessels from the host. If the scaffold is acellular, then the primary disadvantage is the limitation of the transplanted tissue to form anything but fibrovascular scar tissue.

The third approach involves pre-vascularizing a tissue construct prior to implantation. This approach holds the most long term potential because the physical dimensions of the implantable tissue can become much larger. Although this approach must eventually address the robust immunological response to non-autologous endothelial cells recent reports suggest that stem cells and endothelial cells can be easily collected from peripheral blood, Balconi G, Spagnuolo R and Dejana E., “Development of endothelial cell lines from embryonic stem cells: A tool for studying genetically manipulated endothelial cells in vitro,” Arterioscler Thromb Vasc Biol 20: 1443-1451, 2000; Fontaine M, Schloo B, Jenkins R, Uyama S, Hansen L and Vacanti J P., “Human hepatocyte isolation and transplantation into an athymic rat, using prevascularized cell polymer constructs,” J Pediatr Surg 30: 56-60, 1995; and Shima D T, Deutsch U and D'Amore P A, “Hypoxic induction of vascular endothelial growth factor (VEGF) in human epithelial cells is mediated by increases in mRNA stability,” FEBS Lett 370: 203-208, 1995 providing a possible solution for the design and production of an implantable vascularized tissue from autologous cells. Alternatively, induced tolerance may allow the immune response to be sufficiently attenuated for a transplant to succeed. Nonetheless, very little work has been pursued in this area due to the technical challenges of developing a stable vascular network in vitro. One strategy is to prevascularize a tissue by implanting a degradable polymeric construct into a host allowing a fibrovascular tissue to develop. Then, to inject cells specific to the tissue function of interest (in this case hepatocytes) Fontaine M, Schloo B, Jenkins R, Uyama S, Hansen L and Vacanti J P., “Human hepatocyte isolation and transplantation into an athymic rat, using prevascularized cell polymer constructs,” J Pediatr Surg 30: 56-60, 1995. This strategy is again limited by the need for ingrowth of new vessels into the initial polymer construct.

The use of micromachining or microfabrication (also loosely called micro-electro-mechanical systems, MEMS) techniques to assist in the design of engineered tissues has been reported in the design of a vascular network. Bhatia S N, Yarmush M L and Toner M., “Controlling cell interactions by micropatterning in co-cultures: hepatocytes and 3T3 fibroblasts,” J Biomed Mater Res 34: 189-199, 1997; Chen C S, Mrksich M, Huang S, Whitesides G M and Ingber D E., “Micropatterned surfaces for control of cell shape, position, and function,” Biotechnol Prog 14: 356-363, 1998; Chiu D T, Jeon NL, Huang S, Kane R S, Wargo C J, Choi I S, Ingber D E and Whitesides G M, “Patterned deposition of cells and proteins onto surfaces by using three-dimensional microfluidic systems,” Proc Natl Acad Sci USA 97: 2408-2413, 2000; Folch A, Jo B H, Hurtado O, Beebe D J and Toner M., “Microfabricated elastomeric stencils for micropatterning cell cultures,” J Biomed Mater Res 52: 346-353, 2000; Ito Y., “Surface micropatterning to regulate cell functions,” Biomaterials 20: 2333-2342, 1999.

In one case, a two-dimensional template was created to resemble a branching vascular pattern. Endothelial cells were then grown to confluence in this pattern, then lifted and rolled into a three dimensional form. Although very interesting, this study was not able to demonstrate perfusion of the engineered capillary network through connection at the engineered network to the endogenic vasculature. Kaihara S, Borenstein J, Koka R, Lalan S, Ochoa E R, Ravens M, Pien H, Cunningham B and Vacanti J P, “Silicon micromachining to tissue engineer branched vascular channels for liver fabrication,” Tissue Eng 6: 105-117, 2000.

The clinical demand for a transplantable thick tissue with controllable dimensions and mechanical properties is enormous. Every year in the U.S. there are more than one million (1.3 million in 2000) reconstructive surgeries, with most procedures limited to autologous tissue transfer. The most common procedures include repair of tissue following tumor removal, hand surgery, reconstructive breast surgery following partial or total mastectomy, and repair of laceration.

Another large market for vascular tissue is to repair poststernotomy mediastinitis (infection in the mediastinum) following cardiothoracic surgery. Approximately 500,000 open heart surgeries are performed every year, and 1-2% of these are complicated by mediastinitis. Current therapy utilizes vascular tissue flaps, which incur additional time and risk in the operating room, as well as donor site morbidity. Health care costs related to donor site morbidity and length of operation are significant, and could be considerably reduced if a pre-vascularized thick tissue construct were available. Perhaps an equally critical need for a vascularized tissue construct is to minimize post-implant infection. The majority of donor sites are undesirable for artificial implants being either avascular or unsterile. A vascular supply is the only means of delivering the normal host immune response or exogenous antibiotics. Hence, the most important factor limiting the design of thick artificial tissues, thus obviating autologous donation, is an in vitro vascular supply to maintain the artificial tissue upon transplant.

### SUMMARY OF THE INVENTION

Tissue engineering holds enormous potential to replace or restore function to a wide range of tissues. As noted above, most applications have been in thin (<2 mm) avascular tissues in which delivery of nutrients and oxygen occurs primarily by diffusion. The design of more complex organs such as the heart, lung, or thicker connective tissues (>1 cm³) will require a vascular network similar to that in vivo to deliver oxygen and essential nutrients. The successful design of thick three-dimensional vascular tissues requires rapid delivery of oxygen and essential nutrients upon implantation, and thus depends on the creation of a vascular network in vitro prior to implantation.

Pursuant to this invention, a temporary biodegradable microfluidic network is created in an engineered tissue construct. The microfluidic network performs multiple functions. The network supplies essential nutrients to sustain a developing capillary network in vitro, to form fluid connections known as an “anastomoses,” with the endogenous vascular network, to supply oxygen to sustain the tissue and to permit therapeutic compounds, endogenous wound-healing, and infection fighting cells, to enter the transplanted tissue. The creation of such a network in vitro creates an ideal transplant construct to integrate into a patient's host tissue. The capability to perform all of the above functions and more increases the usefulness of the construct of the invention, and as described herein, allows the construct to be larger, thicker and have stronger mechanical properties such that the construct can be used with a wide variety of endogenous tissues, and in a wide variety of surgical applications.

Moreover, once the tissue construct is implanted, and the engineered vascular network of the construct and the endogenous vasculature of the patient anastamose to provide an adequate blood supply to maintain viability of the tissue construct, it is preferred that the microfluidic network biodegrade so that the vasculation of the construct becomes functionally and structurally indistinguishable from the patient's own system.

Therefore, the tissue construct of the invention satisfies at least three basic functional requirements, and these three requirements also reflect three important method steps for producing the tissue construct in practice. Referring to FIG. 1, first, an in vitro vascularization of a tissue construct is created using a biodegradable microfluidic network for delivery of oxygen nutrients. Second, the tissue construct is transplanted to the endogenous wound bed where vessels of the tissue construct and the host rapidly anastomose. Third, host-directed remodeling and reorganization of the tissue and vascular network is seen. In combination with this last step, the biodegradable network is allowed to dissolve as the result of the materials selected and the engineered structure and orientation of the microfluidic network. The fabrication of biodegradable microfluidic channels in a tissue construct to supply nutrients and oxygen to a developing network of endogenous capillaries in a patient is further described in the Examples below.

Pursuant to this invention, a biodegradable (temporary) artificial network of channels delivers essential nutrients and oxygen to the interior portions of a prevascularized thick tissue. Pursuant to this invention, the vascular supply of the tissue is developed in vitro, and thus overcomes the limitations of other strategies that rely on ingrowth of new vessels.

Also, rather than dictating the vascular branching pattern and geometry, the vascular network in the tissue constructs of the invention develops naturally based on intrinsic biological signals, and the microfabrication technology described herein carefully controls the delivery of essential nutrients and oxygen in a temporary biodegradable microfluidic network.

### DESCRIPTION OF THE FIGURES

FIG. 1 shows the overall scheme for generating a prevascularized thick tissue that involves three steps. Step 1: Thick prevascularized tissue constructs are generated in vitro using a biodegradable (or temporary) microfluidic network to deliver essential nutrients. Step 2: Upon implantation to a prepared wound bed, the capillary network of the prevascularized tissue rapidly anastomoses to the host vasculature. Step 3: The temporary microfluidic network degrades over time in vivo leaving only the blood-perfused tissue implant in the host.

FIGS. 2A-2D show the sprout of endothelial cells in collagen gels to form anastomosing capillary networks. (a) 1 day after embedding in the gel, EC sprouts can be seen emerging from the surface of the cytodex bead. (b) 3 days later, sprouts are elongated and branching. (c) By 5-7 days, sprouts are anastomosing with sprouts from adjacent beads—a branched network is formed. (d) hematoxyln-stained sections reveal sprouting vessels composed of multiple cells, and with patent lumens.

FIG. 3 shows how in vitro capillary network is defined by two physical dimensions and capillary network is characterized by three criteria. In the left panel, the in vitro capillary network is characterized by three critical dimensions: 1) C—the distance separating the capillary network from the nutrient media, 2) F—the distance separating the fibroblast monolayer from the nutrient media, and 3) A—the distance separating the fibroblast monolayer from the capillary network. In the right panel, the capillary sprouts from a single bead, prior to anastomosing with vessels from a neighboring bead, can be characterized by three criteria: 1) number of vessel sprouts which can be traced back to the bead (black vessels or vessels #1-#4), 2) total number of individual vessels (black plus red or vessels #1-#6), and 3) total length of all vessels (cumulative length of vessels #1-#6).

FIG. 4 shows that a diffusion limit exists for the delivery of nutrients and mediators from both the growth media and fibroblasts to the capillary network. Total vessel length of the capillary network is shown as a function of several dimensions in the in vitro system—1) the distance separating the capillary network from the nutrient media, 2) the distance separating the fibroblasts from the nutrient media, F, and 3) the distance separating the fibroblasts from the capillary network, Δ.

FIG. 5 shows images of a vascular network with increasing depth of tissue qualitatively demonstrating the presence of a diffusion limitation. Representative images are shown of beads with the capillary networks that were quantified in FIG. 4. For each panel, the distance separating the fibroblasts from the beads is held constant at 1.8 mm and the distance separating the capillary network from the nutrient media (C) progressively increases from 3.6 to 8.1 mm (bold-face number below each panel). Note the qualitative decrease in the number of vessels once C reaches approximately 5 mm.

FIG. 6 shows that the transplanted, in vitro-derived capillary beds have anastomosed with host vasculature. In the left panel, an H&E-stained section showing human in vitro-derived capillaries within a collagen gel, inserted under a skin flap on the back of a SCID mouse. Note that the capillaries are filled with blood. In the right panel, the same tissue is stained with human-specific anti-factor VIII antibody, indicating that the vessels are indeed of human origin.

FIG. 7 shows water-soluble sacrificial substrate that is micro-machined on micron length scale. Microfabricated gelatin rows on a PDMS substrate (top panel) as viewed from above with brightfield microscopy. The rows are approximately 100 microns in diameters, 55 microns in height, and 450 microns apart (bottom panel) as determined by a surface profilometer. The rows wee fabricated by combination of soft lithography and micromolding in capillaries (MIMIC). Dimensions of the rows can be manipulated between 10-200 microns in width and height.

FIG. 8 shows how microfabrication technology can generate biodegradable fluidic channels. A microfabrication scheme to design biodegradable (temporary) microfluidic channels of controlled dimensions and spacing is shown. Lower images are cross-sections at point A-B. In Step 1 (first panel), a thin coating of PLGA is established. In Step 2 (second and third panels), MIMIC is utilized to generate a rectangular water-soluble (sacrificial) positive relief, which forms the dimensions of the channel lumens. In Step 3 (fourth panel), the positive relief is coated with a thin layer of PLGA which forms the top and sides of the channels. In Step 4 (fifth panel), the water soluble substrate is removed by dissolution leaving the patent microfluidic channels as formed.

FIGS. 9A and 9B is a schematic for combining the microfluidic channels with the EC coated cytodex beads in a collagen-fibrin gel. FIG. 9A is a schematic for connecting the microfluidic network to a syringe pump via polyethylene tubing for external delivery of culture media. Once the gelatin micropattern is formed on a substrate, small diameter polyethylene tubing is placed around inlet and outlet. A poly(dimethlysiloxane) (PDMS) mold will act as a barrier, and will be placed around the microfluidic network and a layer of PLGA is applied to form the membrane on top of patterned gelatin (Step 4 of FIG. 6). The gelatin is dissolved away by application of water, yielding hollow microchannels of PLGA. In FIG. 9B, following completion and assembly of the final device, EC-coated Cytodex beads in collagen-fibrin gel will be poured into the reservoir created with the PDMS barrier. As the microfluidic network is perfused with cell culture media with growth factors, oxygen and other nutrients as well as soluble growth factors will pass across the PLGA membrane and reach the ECs.

FIG. 10 shows that microfluidic and capillary networks are stacked to achieve greater depth. Once the feasibility of the single microfluidic network is established, stacking microfluidic and capillary networks achieves tissue depth as shown schematically in the Figure. The flow of media can be countercurrent to minimize nutrient concentration gradients. The spacing between the “layers” will be a key design parameter in this strategy as a means to manipulate final tissue dimensions

FIGS. 11A and 11B illustrates photolithography and MIMIC are used to generate a sacrificial gelatin pattern. Photolithography is used to generate a PDMS mold (FIG. 11A) that can then be used as the template to generate a desired three-dimensional sacrificial structure of gelatin using micromolding in capillaries (MIMIC) (FIG. 11B).

FIG. 12A-12C are a schematic of experimental protocol to determine diffusion limits of nutrients and soluble factors from the growth media and the fibroblast. (FIG. 12A) Fibroblast monolayer separates the endothelial growth media (EGM) from the capillary network. The distance of the capillary network from the media is C, the distance of the fibroblast from the media is F, and the distance separating the fibroblasts from the capillary network is Δ. (13). The capillary network separates the EGM from the fibroblast, and in this orientation C and Δ are held constant and F has increased allowing the relationship between the capillary network and F to be established. A similar scenario can be established for holding F and Δ constant to determine the impact of C. The impact of Δ can be determined once the impact of F and C are known as described in the text. (C) The fibroblasts are cultured separately and the media from this culture is added to the EGM to create a conditioned media The distance of the capillary network from the media is denoted C*.

FIG. 13 is a schematic depicting the implantation of an avascular tissue construct. An avascular tissue construct is constructed in vitro in which a fibroblast monolayer is placed a fixed distance, F, from the nutrient media. The fibrin-fibroblast tissue is within a rigid implantable and impermeable (on all sides except the top) “container.” This container and fibrin-fibroblast tissue is then covered with a semi-permeable polycarbonate membrane (either 0.4 or 3.0 micron pore) on the top surface and then implanted into a subcutaneous pouch of a SCID mouse. The semi-permeable membrane limits ingrowth of host vessels and thus maintains a constant value of F. The semi-permeable membrane can also be omitted to determine the impact of the ingrowth of host vessels. After 3, 7, or 21 days, the animal is sacrificed and the implanted tissue removed to determine extent of vessel ingrowth (if semi-permeable membrane is omitted) and fibroblast viability. In this fashion, a maximum attainable dimension F for viable fibroblasts is determined and compared to that attained with the prevascularized tissue.

FIG. 14 is a schematic depicting the implantation of a pre-vascularized tissue construct. A pre-vascularized tissue is constructed in vitro in which a fibroblast monolayer is placed a fixed distance, F, from the nutrient media, and the capillary network separates the fibroblasts from the media. The pre-vascularized tissue is within a rigid implantable and impermeable “container.” This container and prevascularized tissue is then implanted into a subcutaneous pouch of a SCID mouse. After 3, 7, or 21 days, the animal is sacrificed and the implanted tissue removed to determine extent of vessel ingrowth and fibroblast viability. Dimensions C and D can be manipulated with the goal of attaining the maximum dimension F, which can then be compared to that attained with the avascular tissue.

### DETAILED DESCRIPTION OF THE INVENTION

### EXAMPLE 1

### In Vitro Formation of Vascular Networks

To provide the tissue construct of the invention with sufficient oxygenation, nutrient delivery and overall viability, endothelial cells [EC] can be induced to form complex, anastomosing capillary-like networks in vitro. The networks generated are stable (no apopotosis), exhibit long-term survival (several weeks), and readily anastomose to networks of capillaries with patent lumens. By controlling gel pH at approximately 7.4 (which affects rigidity), growth factor (VEGF, bFGF) concentrations (which affects vessel length and diameter), bead concentration at approximately 200 beads per ml of tissue (which affects degree of anastomosis and complexity of the network) and gel composition at approximately 2.5 mg/ml of Fibrin (the presence of fibronectin stimulates sprouting) capillary network formation is optimized.

Briefly, human umbilical vein endothelial cells are harvested and passaged twice, then seeded onto 150 micron diameter Cytodex beads. The cell-coated beads are then placed in a 2.5% Fibrin gel at the bottom of a 12-well plate (1 cm diameter well) with appropriate growth factors (i.e., VEGF) and a monolayer of fibroblasts a fixed distance (order mm) away. Fibroblasts condition the medium with growth factors such as angiopoietin-1 that stabilize newly-formed vessels. Interestingly, the capillaries are not invested with support cells, but do appear to be stabilized by fibroblast-derived factors, suggesting a direct effect rather than an indirect effect through pericytes as has been proposed in vivo. Directly embedding EC in fibrin gels does not yield a similar network, which probably reflects the need for EC to go through the full program of sprouting, migration, alignment and tube formation, rather than being forced to “coalesce” into a vessel from an initial random distribution of cells in the gel.

Referring to FIGS. 2A-2D, after 1-2 days EC sprouts begin to form. These rapidly elongate over the next couple of days and begin to branch (FIG. 2B). By 5-7 days anastomosing networks, often connecting two beads, are clearly apparent (FIG. 2C). All the vessels are lined by multiple EC, surrounding clear lumens (FIG. 2D). A series of prevascularized tissues constructs following the protocol described briefly above and manipulated several key physical dimensions to determine whether the diffusion limitation of essential nutrients in vitro could be identified and overcome. In vitro size limitations, as well as the relative importance of distances separating the capillary network and fibroblast monolayer from the nutrient media (as from each other), must first be established before determining the upper limit on physical dimensions in vivo.

Referring to FIG. 3, FIG. 3 (left) depicts a schematic of a well plate with the prevascularized tissue and three key physical dimensions: 1) C—the distance separating the capillary network from the nutrient media, 2) F— the distance separating the fibroblast monolayer from the nutrient media, and 3) A—the distance separating the fibroblast monolayer from the capillary network. These three criteria are shown schematically in FIG. 3 (right). In this embodiment, the fibroblast layer always separated the capillary network from the media. As C was varied from 3.6 to 8.1 mm, A was held constant at 1.8 mm, but F also necessarily increased from 1.8 to 6.3 mm. Thus, although this experiment cannot decouple the impact of F and C, it can establish the presence of a diffusion-limitation of essential nutrients from the media.

In addition, A was varied from 1.8 to 4.5 mm while holding C constant at 5.4 mm. In this experiment, F was necessarily varied between 0.9 to 3.6 mm and thus this experiment cannot decouple the effect of F and Δ. However, as Δ increases, F decreases and thus the observed diffusion limitation is likely a sole effect of Δ. This analysis provides evidence for a diffusion limitation of soluble mediators from the fibroblast to the capillary network. When C and F exceed 4.5 and 3.6 mm, respectively, there is a significant decrease in the total number of capillary vessels (blue line) suggesting the diffusion limit for essential nutrients from the media has been reached. When A exceeds 2.7 mm (or when F is less than 2.7 mm, however, a smaller F does not approach a diffusion limit), there is also a significant decrease in the number of vessels, also suggesting a diffusion limit for soluble mediators from the fibroblast. The vascular networks were allowed to develop for 6 days, and then a minimum of 2 beads were imaged with low-power (10×) Brightfield microscopy and saved as high resolution (.tif) resolution images for analysis with ScionImage. The vascular networks were then characterized using three criteria or endpoints for each bead: 1) number of vessels sprouts defined as the number of distinct vessels whose length was a minimum of one microsphere radius and whose origin could be traced back to the microsphere, 2) total number of vessel segments, and 3) total length of vascular network defined as the sum of the lengths of all vessels segments. Of note is the fact that only beads exhibiting no anastomoses with neighboring beads were quantified.

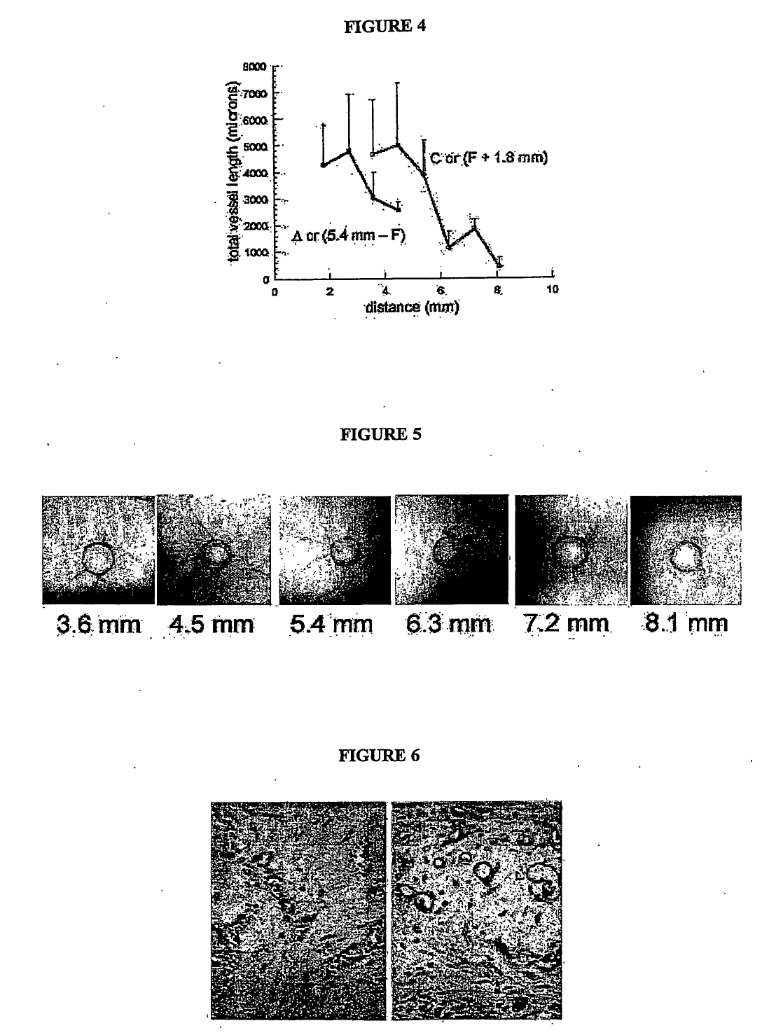

All three endpoints demonstrated a similar trend with the distances (C, F, and A), and FIG. 4 shows only the trend of the total length of the vascular network. As noted above, for each depth, there exists a point at which the total number of vessels begins to decline—4.5 mm for C (or 3.6 mm for F), and 2.7 mm for Δ. FIG. 5 demonstrates the impact of C qualitatively by showing representative beads use in the quantitative analysis of FIG. 4. FIG. 5 shows images of a vascular network with increasing depth of tissue qualitatively demonstrating the presence of a diffusion limitation. Representative images are shown of beads with the capillary networks that were quantified in FIG. 4. For each panel, the distance separating the fibroblasts from the beads (refer to FIG. 3) is held constant at 1.8 mm and the distance separating the capillary network from the nutrient media (C) progressively increases from 3.6 to 8.1 mm (bold-face number below each panel). Note the qualitative decrease in the number of vessels once C reaches approximately 5 mm. From this result, the existence of a maximum physical dimension is demonstrated, and also an optimal physical separation of the fibroblasts from the developing capillary network is quantified.

### EXAMPLE 2

### Transplanted, In Vitro-Derived Capillary Beds Anastomose with Host Vasculature

A crucial concern in transplanting a vascularized tissue into a host is whether the two vascular networks will “hook-up” correctly, allowing perfusion of the transplanted vessels. In two different systems that transplanted vascular beds in tissue constructs spontaneously anastomose with host vasculature. In the first, human epidermis, including the superficial vascular plexus, was transplanted onto the back of an immunocompromised (SCID) mouse. After only three days, anastomoses between human and mouse vasculature could be identified using species-specific antibodies, and moreover, the skin survived through perfusion of human vessels, not through ingrowth of host vessels. Referring to FIG. 6 in the left panel, an H&E-stained section showing human in vitro-derived capillaries within a collagen gel, inserted under a skin flap on the back of a SCID mouse. Note that the capillaries are filled with blood. In the right panel, the same tissue stained with human-specific anti-factor VIII antibody, indicating that the vessels are indeed of human origin.

Under such circumstances full perfusion would take several days during which the thin, cell-poor epidermis could have survived by diffusion of nutrients, an unlikely outcome for thicker tissues. In the second assay, normal (or genetically modified) human endothelial cells were grown in collagen gels before implantation into a skin pocket on the back of a SCID mouse. Schechner J S, Nath A K, Zheng L, Kluger M S, Hughes C C, Sierra-Honigmann M R, Lorber M I, Tellides G, Kashgarian M, Bothwell A L and Pober J S., “In vivo formation of complex microvessels lined by human endothelial cells in an immunodeficient mouse,” Proc Natl Acad Sci USA 97: 9191-9196, 2000. Again, human and mouse vessels anastomosed and mouse erythrocytes could be seen in the lumens of the human vessels, indicating blood flow and demonstrating that endothelial cells grown in culture and induced to reform vessels, still retain the ability to anastomose with host vasculature and form normal, patent vessels.

### EXAMPLE 3

### Manipulation of Gelatin at Micron Dimensions

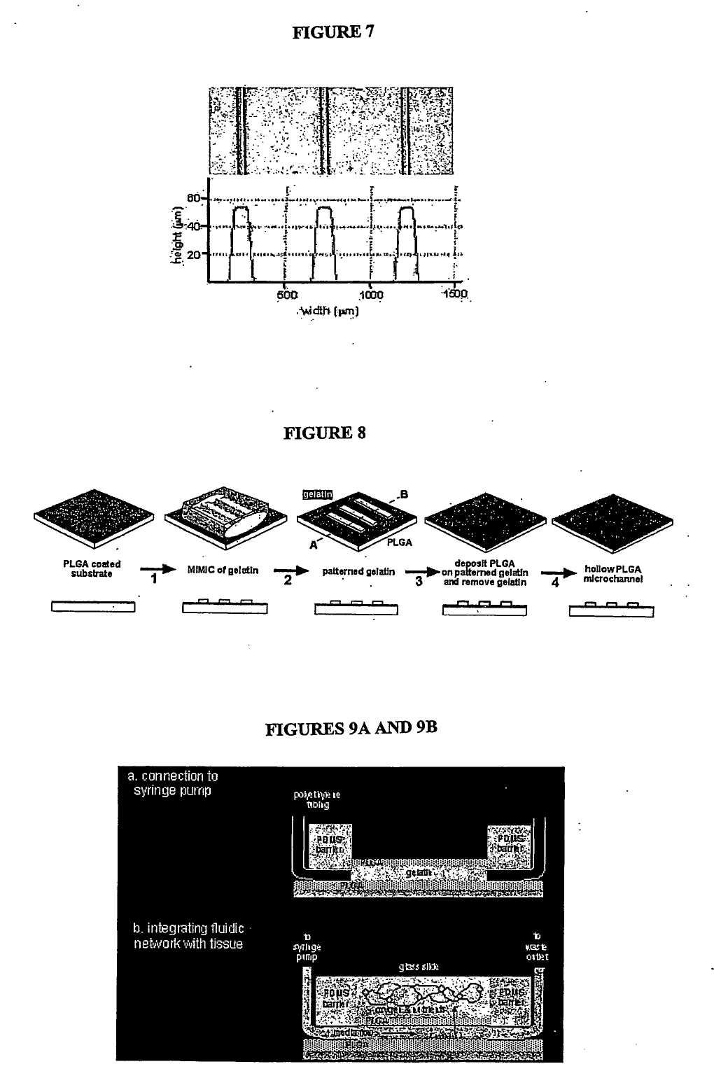

A combination of soft lithography and micromolding in capillaries (MMIC) generates rows of gelatin with dimensions on the micron scale. A high-resolution photomask is generated and used to selectively expose photoresist by contact photolithography. The unexposed photoresist is removed leaving a positive relief that serves as the master mold. Prepolymer of poly(dimethlysiloxane) (PDMS) is cast on the master and cured to obtain a PDMS replica with embedded channels. A solution of gelatin (appropriate viscosity) is then wicked into the PDMS mold by capillary action MIMIC) and allowed to gel. The PDMS mold is removed leaving the micropatterned gelatin rows (FIG. 4). Referring to FIG. 7, the rows are approximately 100 microns in diameter, 55 microns in height, and 455 microns apart (bottom panel) as determined by a surface profilometer. The gelatin rows serve as the mold for the lumen of the microfluidic channels. Following coding with PLGA, the gelatin is removed by dissolution leaving the patent fluidic channels. The dimensions of the rows are easily manipulated between 10-200 microns depending on the master mold and the viscosity of the gelatin solution.

### EXAMPLE 4

### Porous Microfluidic Network with Controlled Channel Diagrams, Porosity Degradation Rate and Volumetric Flows

In a preferred embodiment, parameters such as channel dimensions (width, height, and length), wall thickness, channel spacing, porosity, volumetric flow rate, and degradation rate are controlled by the thickness of the fluidic channels and the polymer composition. Referring to FIG. 8, the overall steps include: 1) depositing a two-dimensional thin film of a degradable polymer (e.g., PLGA) which will serve as the base of the rectangular channels; 2) patterning a network of lines/features from a water-soluble sacrificial material (fish gelatin, solid at room temperature) as the next layer, which will serve as the mold for the channel lumens; 3) depositing another layer of the degradable polymer over the network of water-soluble material by solvent casting; 4) removing the water soluble material by dissolution leaving the hollow network of degradable microfluidic channels.

The fabrication of the hollow, degradable microchannels is carried out on a substrate coated with a thin layer of PLGA. The thickness of this bottom layer is controlled by using varying amount of PLGA dissolved in the solvent (CH₂Cl₂) and controlling the spin speed (spin casting will be used to obtain a thin uniform layer).

Micromolding in capillaries (MIMIC), Xia Y and Whitesides G M., “Soft Lithography,” Angew. Chem. Int. Ed. 37: 550-575, 1998, is used to pattern a sacrificial layer (water soluble gelatin that is solid at room temperature) that is removed to yield hollow microchannels. In the MIMIC process, the PDMS (define) mold is placed on the surface of a substrate and makes conformal contact with the substrate. The relief structure in the mold forms a network of hydraulically-connected empty channels. When a solution of gelatin is placed at the open end of the network of channels, the liquid spontaneously fills the channel by capillary action. After filling the channels, the polymer is dried in an oven. When the PDMS mold is removed after the polymer is cured, a pattern of gelatin remains on the substrate.

After patterning, the gelatin is coated with a thin layer of PLGA. The coating procedure should yield a uniform layer around the sharp edges of the gelatin channels. Since the thickness of the degradable PLGA will affect the diffusion of nutrients and oxygen to the surrounding tissue, coating thickness must be uniform and free of macroscale defects that would result in leakage.

### EXAMPLE 5

### Microfluidics are Combined with Endothelial Cells

Following generation of the microfluidic network, the collagen-fibrin gel containing endothelial seeded Cytodex beads is poured into a well lined on the bottom by the microfluidic network and on the sides by a PDMS mold. Referring to FIGS. 9A and 9B, the surface of the collagen-fibrin Cytodex bead matrix will then be covered by a glass slide to serve as an oxygen impermeable boundary. A syringe pump will control the flow rate of fluid through the network of biodegradable channels. The connection from the syringe pump to the fluidic channels will be a polyethylene tube that will be incorporated at the time of fluidic channel fabrication as detailed in FIG. 9A. Once the gelatin micropattern is found on a substrate, the small diameter polyethylene tubing is placed around the inlet and outlet. A poly(dimethylsiloxane)(PDMS) mold acts as the barrier and is placed around the microfluidic network and the layer of PLGA to form the membrane on top of the patterned gelatin. After the final device is completed and assembled, EC-coated Cytodex beads and collagen-fibrin gel are poured into the reservoir created with the PDMS barrier. The microfluidic network is profused with cell culture media with growth factors, oxygen, and other nutrients as well as celluble growth factors that pass across the PLGA membrane and reach the ECs. Flow rates between 100 manoleters per minute to 1 ml per minute provide effective maintenance of endothelial cell for viability.

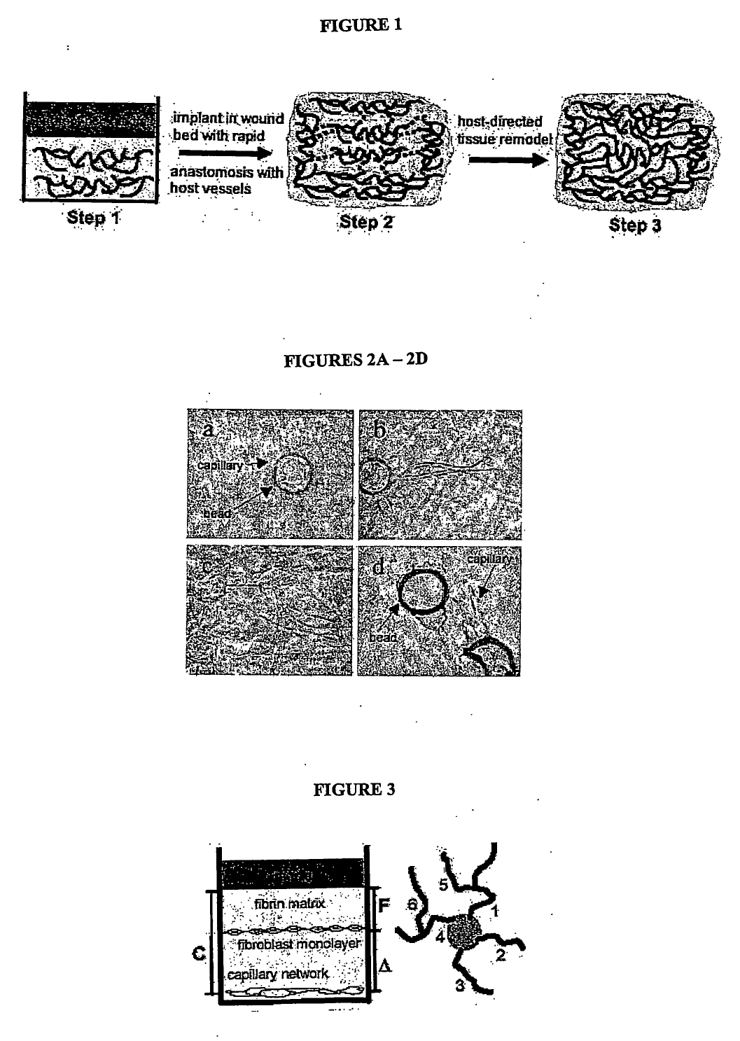

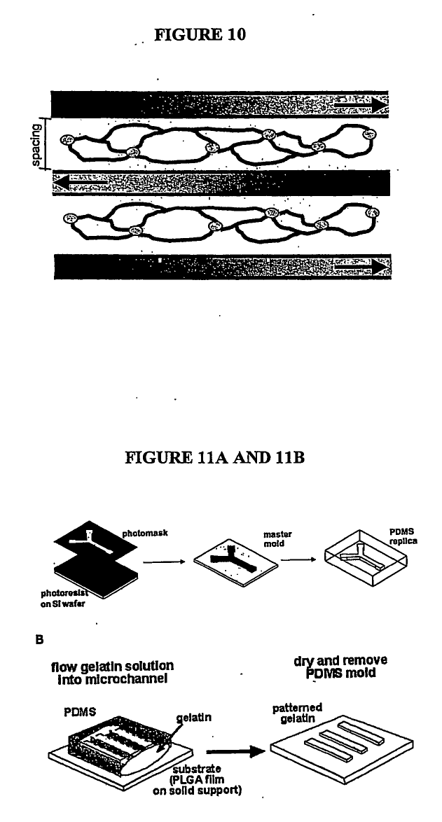

The first “layer” of the microfluidic network and capillary bed is a critical element. Once established, additional layers are added to create depth to the tissue as first described in FIG. 1. FIG. 10 depicts a scenario in which microfluidic and capillary networks are stacked (in layers), and the flow of nutrients is in a countercurrent fashion. This embodiment is desirable to minimize nutrient gradients within the tissue.

### EXAMPLE 6

### Microfabrication

The fabrication of gelatin and PLGA microchannels is performed using photolithography and soft lithography (MIMIC, micromolding in microcapillaries, a form of microfabrication adopted for biological applications). FIG. 11A and 11B show the schematic of the process for fabricating PDMS mold for MIMIC by photolithography. A CAD program is used to design a pattern of microchannels from which a high-resolution photomask is generated. This photomask is used to selectively expose photoresist by contact photolithography. Developing away the unexposed photoresist leaves a positive relief that can serve as a master mold. Prepolymer of PDMS is cast on the master and cured to obtain a PDMS replica with embedded channels.

Micromolding in capillaries (MIMIC) is an alternative to photolithography that can pattern biological materials (gelatin and collagen) and molecules (proteins). A variety of materials have been patterned using MIMIC including photopolymers, ceramics, beads, inorganic salts, biological macromolecules as well as cell suspension. In MIMIC, the PDMS mold is placed on the surface of a substrate and makes conformal contact with the substrate. The relief structure in the mold forms a network of empty channels. When a drop of the gelatin solution (diluted with PBS to appropriate viscosity) is placed at the open end of the network of channels, the liquid spontaneously fills the channel by capillary action. After filling the channels, the gelatin solution is dried for 30 min at 50° C. to cure and solidify. When the PDMS mold is removed, a pattern of solid gelatin is patterned on the substrate.

One of two well-characterized biodegradable polymers is used for in the synthesis and characterization of biodegradable microspheres: 1) Poly-1-lactide-poly-glycolic acid (PLGA), and 2) Polyethylene glycol-poly-1-lactide (PELA). The biodegradability and mechanical strength of both polymers is controlled with molecular weight and composition of hydrophilic species as detailed below.

PLGA is commercially available in a wide molecular weight range (8-140 KD), and lactide/glycolide ratio (0-46% (w/w) glycolide) from Alkermes, Inc. (Cincinnati, Ohio). The higher molecular weight polymer is expected to have higher mechanical strength, and a slower degradation rate in an aqueous phase. The degradation rate varies from two weeks to sixteen months depending on the lactide/glycolide ratio, and the molecular weight of the polymer. The glycolide content determines the hydrophilicity of the polymer chain; thus, since degradation occurs due to hydrolysis, increasing the glycolide content will increase the degradation rate.

PELA is more hydrophilic than PLGA, and is synthesized from polyethylene glycol (PEG) and 1-lactide monomer in the presence of a small amount of catalyst such as stannous 2 ethyl-hexanoate at high temperature (180° C.). PEG content (5-10%) in the polymer can be controlled from the initial content of PEG during the polymerization reaction. The presence of PEG enhances hydrophilicity of the polymer chain because of its strong hydration property, thus, increasing the PEG content will increase the rate of degradation.

### EXAMPLE 7

### Endothelial Cell Culture and Capillary Network

Microvascular, or umbilical vein, EC are grown to confluence and then harvested and mixed with collagen-coated Cytodex beads at a ratio of 400 cells per bead. This mixture is then cultured for 4 hours with gentle mixing every 30 min. The beads and cells are then cultured overnight in uncoated culture flasks. Beads are harvested and mixed with 2.5 mg/ml fibrinogen at a density of 200 beads per ml and thrombin (0.625 U/ml) is added. After clotting, fibroblasts are plated to confluence on top of the gel and medium, aprotinin and growth factors (VEGF and bFGF) are added and the plates cultured at 37° C.

Although this culture system for capillary network formation has been optimized for vessel growth, patency, and stability while receiving nutrients from passive diffusion, the following parameters can be manipulated to optimize the culture conditions when nutrients are delivered by the microfluidic network: bead density, bead size, fibrinogen and thrombin concentration, endothelial cell seeding density, concentration of growth factors, density, vessel length and diameter, number of branch points and interbranch distance. A combination of manual and automated (computer-assisted) morphometric techniques are used to quantify the various network parameters. From these data, mean intercapillary distances are compared to known values for various tissues, and predicted maximum distances for maintaining high enough oxygen tension for cell survival.

A fiber optic oxygen sensor (FOXY, Ocean Optics Inc.) is used to measure oxygen levels at various depths in the tissue. The fiber optic probe uses fluorescence quenching technology where the collision of an oxygen molecule with a ruthenium complex excited by an LED leads to an energy transfer without producing heat. The degree of fluorescence quenching correlates to the level of oxygen concentration or to oxygen partial pressure. The probe is mounted onto a modified Nikon TE200 microscope with a computerized stage for precise depth analysis. The concentration, spatial and temporal resolution of this system is anticipated to be ˜0.02 ppm, 10 microns, and <50 msec, respectively.

Other oxygen measurement systems are available such as Clark-style electrodes and phosphorescence decay devices. Clark-style electrodes can drift due to stretching and protein fouling of the PTFE membrane which slows oxygen permeation. In addition, the Clark electrodes consume oxygen making interpretation of the measurements more difficult Phosphorescence oxygen measurement systems require phosphor diffusion into the tissue. This method works well when the phosphor is injected into the blood system but diffusion through tissue in vitro is less effective.

### EXAMPLE 8

### Quantitation of Diffusion Limits (Maximum Physical Dimensions) for Fibroblast Cell Survival and Capillary Network Formation in an Existing In Vitro Model of Angiogenesis

The existence of a diffusion limitation for nutrients from the media and soluble factors from the fibroblast may limit the healthy development of the capillary network in vitro. Also, diffusion limits could limit the physical dimensions attainable in vitro prior to implantation, and could impact the rationale design of the tissue including such critical information as separation distance between the fibroblast and the capillary network. In addition, this information is needed to determine whether the in vitro or in vivo environment is more limiting. In the former, nutrients are delivered purely by diffusion and the source is a nutrient rich media. In the later, nutrients are initially delivered by diffusion alone, but ingrowth of host vessels will provide nutrients by convection. In addition, the source of nutrients is initially the plasma exudate in the wound bed and other cell types besides the interstitial fibroblast.

Referring to the parameters of FIGS. 3 and 4 and the accompanying text above, the distance separating the capillary network from the growth media, C, the distance separating the fibroblast from the growth media, F, and the distance of the fibroblasts from the network, □are measured and the impact of the total depth of tissue on the health of the fibroblast and the capillary network is determined.

FIGS. 12A-12C demonstrates how C and F are altered to establish the independent diffusion limitation of nutrients from the media to the capillary network and the fibroblasts. Conceptually, once the impact of C and F is known, any remaining effect on the capillary network can be attributed to Δ. The specific impact of each variable can be addressed more quantitatively using a simple empirical model. Let the total vessel length be equal to L and then assume that L is a function of C, F, and Δ. Then, the following linear model without interaction between the variables would be the simplest approach:
L(C,F,Δ)α+βC+φF+γΔ
where α, β, γand Δ are constants determined using a least squares algorithm. One can then determine the relative importance of each parameter, and whether each parameter has a significant impact by determining whether it is significantly different from zero. Referring to FIGS. 12A-12C, to analyze diffusion limitations in a tissue construct, C, F, and Δ are systematically altered. C and F will each have four different depths (2.0, 4.0, 6.0, and 8.0 mm) and thus Δ will simultaneously have three depths (2.0, 4.0, and 6.0 mm). The capillary network may be located either between the nutrient media and the fibroblasts (C<F) and underneath the fibroblasts (C>F). Because C and F cannot take on the same value, 12 (4×3) different combinations exist for C and F. For analytical purposes, three tissues can be determined at each condition, and 5 beads per tissue analyzed at a single optimal time point (7 days based on preliminary data).

In a second experiment that can be performed in parallel such that the same batch of endothelial and fibroblast cells can be used, the fibroblasts are removed from the fibrin tissue and fibroblast-conditioned media (FIG. 12C) is used C* is altered over the same four depths to determine diffusion limited distances for essential nutrients from the growth media and the fibroblast, and to determine whether the fibroblast must be present in the same culture system as the capillary network.

For each of the experimental conditions, the capillary networks are quantified using the endpoints described above—total length of vessel network, number of vessel sprouts, and number of vessel segments using low magnification, high resolution brightfield images. A diffusion-limited distance is associated with both C and F; however, the relative magnitude of this effect may be different.

It is known that the growth of new capillaries and the health of the fibroblasts depend on different essential nutrients. For example, hypoxia stimulates new capillary growth, Fukumura D, Xu L, Chen Y, Gohongi T, Seed B and Jain R K., “Hypoxia and acidosis independently up-regulate vascular endothelial growth factor transcription in brain tumors in vivo,” Cancer Res 61: 6020-6024, 2001 and Helmlinger G, Endo M, Ferrara N, Hlatky L and Jain R K., “Formation of endothelial cell networks,” Nature 405: 139-141, 2000, but impairs the function of the fibroblast. Mutual or two-way signaling between the endothelial cell and the fibroblast may also be critical for capillary growth, and is expected, thus a different response of the capillary network when only fibroblast-conditioned media is used.

### EXAMPLE 9

### The Diffusion Limits for Tissue Survival (Fibroblast Embedded Fibrin Gel) Following In Vivo Implantation of an Avascular Tissue are Determined for Comparison with the Revascularized Tissue of Example 10 Below

This analysis establishes an in vivo model of an implantable avascular tissue with a well-defined experimental endpoint for assessing tissue viability. A monolayer of fibroblasts embedded within a fibrin matrix is placed at a fixed distance from the matrix-media or matrix-host interface. To control access of nutrients, the bottom and sides of the tissue are made impermeable to the diffusion of nutrients by developing the tissue in vitro within an implantable rigid “container” in the shape of a cylinder. The cylinder is made of a biologically inert material (i.e., will not degrade in vivo) for this experiment such as poly(dimethlysiloxane) (PDMS) or Teflon. In this fashion, the cylinder provides structural support for the fibrin gel and also limits diffusion of nutrients to one area and only one direction.

The cylinder with the fibrin tissue will then be placed in a bluntly dissected subcutaneous pouch on the anterior abdominal wall of 5-8 week old ICR-SCID-beige mice (C.B-17/IcrHsd-scid-bg, Harlan-Sprague-Dawley). The ICR-SCID-beige mouse is outbred, nonleaky, and in addition to lacking T and B cells also lack functioning NK (natural killer) cells. Thus, these mice are unable to amount an immune response to an implanted foreign body or tissue. The wound will be closed with staples and thus the cutaneous layer of tissue will form the “lid” of the cylinder and thus the only source of nutrients to maintain viability of the fibroblast monolayer.

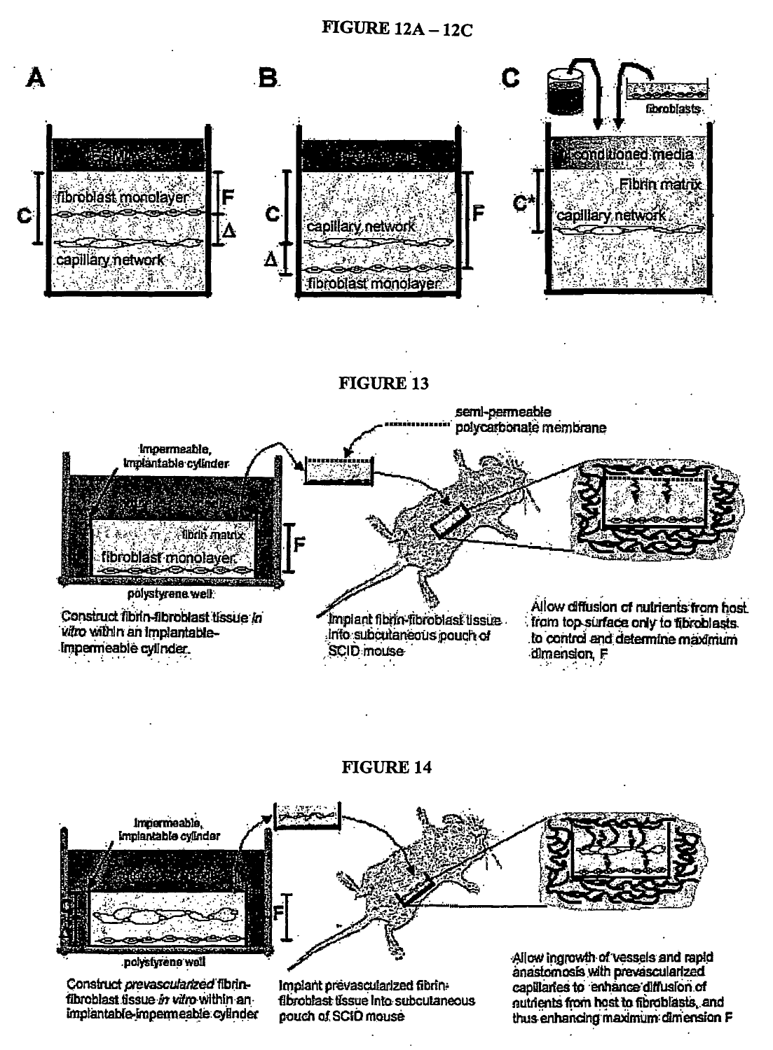

The key experimental variable is the thickness of the acellular fibrin gel overlying the fibroblasts. In each mouse, a maximum of 4 separates tissues can be placed, and thus 4 depths or values of F can be studied simultaneously within the same host. FIG. 13 schematically describes the protocol. Because host vessels are anticipated to penetrate the tissue, a semi-permeable polycarbonate membrane is placed over the top of the tissue implant to limit nutrient access by diffusion only in one-half of the tissues.

The tissues are left in the subcutaneous pouch for either 3, 7, or 21 days, after which the animal are sacrificed and the tissue removed for analysis. Analysis of the tissue includes the following experimental endpoints:
1) Evidence of apoptosis in the fibroblast using the TUNEL staining of the fixed tissue.
2) Evidence of necrosis in the fibroblast monolayer by H&E staining and conventional histology.
3) Evidence of host vessel ingrowth and thus a dynamically changing value for dimension F.

The end points identify a critical distance of separation between the fibroblast monolayer and the cutaneous tissue needed to maintain viability of the fibroblasts. This distance is likely to be on the order of 24 mm based on our in vitro observations and in vivo observations by other groups using alternate implantable models, Loebsack A, Greene K, Wyatt S, Culberson C, Austin C, Beiler R, Roland W, Eiselt P, Rowley J, Burg K, Mooney D, Holder W and Halberstadt C., “In vivo characterization of a porous hydrogel material for use as a tissue bulking agent,” J Biomed Mater Res 57: 575-581, 2001 and Schechner J S, Nath A K, Zheng L, Kluger M S, Hughes C C, Sierra-Honigmann M R, Lorber M I, Tellides G, Kashgarian M, Bothwell A L and Pober J S., “In vivo formation of complex microvessels lined by human endothelial cells in an immunodeficient mouse,” Proc Natl Acad Sci USA 97: 9191-9196, 2000.

### EXAMPLE 10

### Functional Vascular Anastomoses Between the Host and an Implanted Prevascularized Tissue Enhances the Physical Dimensions of Viable Tissue In Vivo

Referring to FIG. 14, the protocol is similar to that described in Example 9. The fibrin-fibroblast tissue will now include a capillary network that separates the nutrient-rich media from the monolayer of fibroblasts.

The tissue is placed in a bluntly dissected subcutaneous pouch on the anterior abdominal wall of 5-8 week old SCID mice as described earlier. The tissue is allowed to integrate with the host, the animal will be sacrificed at 3, 7, or 21 days post-implant, and the tissue implant excised and examined with conventional histology and immunohistochemistry. The key variables C, F, and Δ will be varied as described above. Thus, in each mouse, three experimental conditions exist for one control (avascular) tissue. The following experimental endpoints are determined in each tissue:
1) Evidence of apoptosis in the fibroblast and human endothelial cells using TUNEL staining of the fixed tissue.
2) Evidence of necrosis in the fibroblast monolayer and human endothelial cells by H&E staining and conventional histology.
3) Primary antibody labeling of human endothelial cells (anti-factor VIII Ab, see FIG. 6) to determine if mouse red blood cells are perfusing vessels of human endothelial cell origin.
4) Extent of host vessel ingrowth and host cell migration into the implant. From these end points, the maximum depth of tissue in vitro is determined that can maintain viable tissue upon implantation.

Functional anastomoses (i.e., mouse erythrocytes within vessels of human endothelial cell origin) between the prevascularized implanted tissue and the host are preferably formed within 3 days post-implant. Further, these anastomoses deliver essential nutrients to the fibroblast monolayer and enhance the maximum physical dimension (depth of tissue for this experiment) of the implantable tissue. In a successful implant, by 7 days post-implant, evidence of tissue remodeling in the implant, such as invasion of host cells (i.e., fibroblasts), is observed. Similarly, by 21 days, recruitment of pericytes to the periphery of the implantable vessels to form complex microvessels that begin to resemble arterioles is observed. Schechner J S, Nath A K, Zheng L, Kluger M S, Hughes C C, Sierra-Honigmann M R, Lorber M I, Tellides G, Kashgarian M, Bothwell A L and Pober J S., “In vivo formation of complex microvessels lined by human endothelial cells in an immunodeficient mouse,” Proc Natl Acad Sci USA 97: 9191-9196, 2000.

Although this culture system for capillary network formation has been optimized for vessel growth, patency, and stability while receiving nutrients by passive diffusion in vitro. Culture conditions may be altered to optimize the rate and degree of functional anastomoses upon implantation. To do this, the following parameters can be altered: bead density, bead size, fibrinogen and thrombin concentration, endothelial cell seeding density, and concentration of growth factors (e.g., VEGF, bFGF, aprotinin). In particular, VEGF may be withheld after the initial burst of vessel growth in the first 5-8 days to allow soluble factors from the fibroblast (i.e., ang-1 or bFGF) to stabilize the vessels.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in must be understood as being generic to all possible meanings supported by the specification and by the word itself.

The definitions of the words or elements of the following invention and its various embodiments are, therefore, defined in this specification to include not only the combination of elements which are literally set forth, but all equivalent structure, material or acts for performing substantially the same function in substantially the same way to obtain substantially the same result. In this sense, it is therefore contemplated that an equivalent substitution of two or more elements may be made for any one of the elements in the invention and its various embodiments below or that a single element may be substituted for two or more elements in a claim.

Insubstantial changes from the claimed subject matter as viewed by a person with ordinary skill in the art, now known or later devised, are expressly contemplated as being equivalently within the scope of the invention and its various embodiments. Therefore, obvious substitutions now or later known to one with ordinary skill in the art are defined to be within the scope of the defined elements.

The invention and its various embodiments are thus to be understood to include what is specifically illustrated and described above, what is conceptionally equivalent, what can be obviously substituted and also what essentially incorporates the essential idea of the invention.

We claim:

1. A prevascularized tissue construct for transplantation comprising:
a population of endothelial cells maintained in vitro to form a three dimensional construct, wherein the construct is vascularized with a network of capillaries having patent lumens capable of forming an anastomosis with host vasculature following transplantation.

2. The tissue construct of claim 1 wherein a thickness dimension is greater than 2 mm.

3. The tissue construct of claim 1 further comprising a dispersion of synthetic beads.

4. The tissue construct of claim 3 wherein the concentration of synthetic beads is approximately 200 per ml of tissue.

5. The tissue construct of claim 1 wherein the construct is maintained in media comprised of growth factor.

6. The tissue construct of claim 5 wherein the growth factor is selected from the group consisting of VEGF and bFGF.

7. The tissue construct of claim 1 wherein the network is further comprised of fibroblasts.

8. The tissue construct of claim 7 wherein the fibroblasts are arranged in a monolayer and expresses angiopoeitin.

9. The tissue construct of claim 1 wherein the in vitro conditions include a gel containing fibronectin.

10. A method to produce a tissue construct for transplantation comprising:
seeding a population of endothelial cells into a dispersion;
growing the cells in vitro in the presence of growth factors;
forming a vascularized construct in vitro comprising a network of capillaries having patent lumens capable of forming anastamoses with host vasculature following transplantation.

11. The method of claim 10 wherein the seeding step coats endothelial cells onto synthetic beads.

12. The method claim 10 wherein the growing step is comprised of placing cell-coated beads into a gel.

13. The method claim 12 wherein the gel is comprised of fibronectin.

14. The method claim 12 wherein the network is comprised of fibroblasts.

15. The method claim 14 wherein the fibroblasts are arrayed in a monolayer and produce angiopoeitin.

16. The method claim 10 wherein the growing step is performed for at least 7 days.

17. The method claim 10 wherein the growth factors are selected from the group consisting of VEGF and bFGF.

18. The method claim 10 wherein the step of forming the vascularized construct yields a branched network of vessels.

19. The method claim 18 wherein the network of vessels is comprised of clear lumens surrounded by multiple endothelial cells.

20. A method to transplant a prevascularized tissue construct into a patent comprising:
identifying a host site in the patient;
obtaining a prevascularized tissue construct created in vitro, wherein the tissue construct has a network of capillaries comprised of patent lumens capable of forming anastamoses with the vascularization of the host site in the patient;
transplanting the tissue construct into the host site of the patient such that the capillary network of the prevascularized tissue construct forms anastamoses to vasculature at the target site.
